(19) United States
(12) Patent Application Publication
(10) Pub. No.: US 2004/0180086 A1
(43) Pub. Date: Sep. 16, 2004

(54) Gastro-retentive levodopa delivery form

(76) Inventors: Zebunnissa Ramtoola, Dublin (IE); Kenneth I. Cumming, Loughton (GB); Mary L. Martin, Terenure (IE)

Correspondence Address: COOLEY GODWARD, LLP, 3000 EL CAMINO REAL, 5 PALO ALTO SQUARE, PALO ALTO, CA, 94306, US

(21) Appl. No.: 10/684,058

(22) Filed: Oct. 10, 2003

Related U.S. Application Data
(60) Provisional application No. 60/417,829, filed on Oct. 11, 2002.

(51) Int. Cl. A61K 9/46

(52) U.S. Cl. 424/466

(57) ABSTRACT

Gastro-retentive dosage forms for prolonged delivery of levodopa and carbidopalevodopa combinations are described. The dosage forms comprise a tablet containing the active ingredient and a gas-generating agent sealed within an expandable, hydrophilic, water-permeable and substantially gas-impermeable membrane. Upon contact with gastric fluid, the membrane expands as a result of the release of gas from the gas-generating agent in the tablet. The expanded membrane is retained in the stomach for a prolonged period of time up to 24 hours or more during which period the active ingredient is released from the tablet providing delivery of levodopa to the site of optimum absorption in the upper small intestine.

### CROSS-REFERENCE TO RELATED APPLICATION

The present invention is related to and claims priority to U.S. Provisional Patent Application Serial No. 60/417,829, filed Oct. 11, 2002, which is incorporated herein by reference.

### FIELD OF THE INVENTION

This invention relates to a gastro-retentive formulation of levodopa that delivers the drug in a controlled release fashion to the upper small intestine. It also relates to its method of preparation and a method for treating Parkinson's disease.

### BACKGROUND

Parkinson's disease is a progressive, neurodegenerative disorder of the extrapyramidal nervous system affecting the mobility and control of the skeletal muscular system. Its characteristic features include resting tremor, rigidity, and bradykinetic movements. Current evidence indicates that symptoms of Parkinson's disease are related to depletion of dopamine in the corpus striatum. Administration of dopamine is ineffective in the treatment of Parkinson's disease apparently because it does not cross the blood-brain barrier. However, levodopa, the metabolic precursor of dopamine, does cross the blood-brain barrier, and presumably is converted to dopamine in the brain. Levodopa has been and is one of the most commonly prescribed drug for patients diagnosed with Parkinson's, despite new therapies entering the market. Carbidopa is often administered in combination with levodopa. When levodopa is administered orally, it is rapidly decarboxylated to dopamine in extracerebral tissues so that only a small portion of a given dose is transported unchanged to the central nervous system. Carbidopa inhibits the decarboxylation of peripheral levodopa and does not itself cross the blood-brain barrier nor affect the metabolism of levodopa within the central nervous system. Since its decarboxylase-inhibiting activity is limited to extracerebral tissues, administration of carbidopa with levodopa makes levodopa more available for transport to the brain. Carbidopa reduces the amount of levodopa required to produce a given response by about 75 percent.

Most patients using levodopa experience an initial improvement in their symptoms. However more than 50% of patients will develop fluctuations in response (dyskinesias) in the first five years of levodopa therapy, associated with the end-of-dose effect (wearing off) and the on-off phenomenon. In the former, the dose of levodopa does not last as long as it originally did. This problem can usually be managed by decreasing the dosing interval or switching to the sustained release forms of the drug. With the on-off phenomenon, the patient experiences wide fluctuations in function. Again this can be treated by giving more frequent doses of levodopa or to use the sustained release form of the drug.

There are a number of levodopa products currently available. Sinemet® (Dupont) is a combination of levodopa and carbidopa used for the treatment of Parkinson's disease and syndrome. It is available in immediate release (“IR”) and controlled release (“CR”) formulations. SINEMET® is available in tablet form in 3 strengths; 10 mg carbidopa-100 mg levodopa, 25 mg carbidopa-100 mg levodopa, and 25 mg carbidopa-250 mg levodopa. SINEMET® CR is a sustained release form that is available in two strengths: either 50 mg carbidopa-200 mg levodopa, or 25 mg carbidopa-100 mg levodopa. SINEMET® CR tablet uses a polymeric-based drug delivery system in which the release of carbidopa and levodopa is controlled by the erosion of the polymer. The sustained-release dosage form is designed to release these ingredients over a 4- to 6-hour period. With the sustained release form, the variation observed in plasma levodopa levels is less than that observed with the conventional formulation.

Further extension in plasma profile of levodopa has proved to be difficult using conventional controlled release technologies. One obstacle to achieving such a sustained release form of levodopa is that the drug is absorbed only higher in the upper small intestine. Once the controlled release dosage form travels past the site of absorption, no further productive absorption of levodopa will occur. An oral formulation that would provide a constant delivery or sustained plasma level of levodopa for up to 24 hours or more using conventional SR/CR technologies has not been achieved previously. Therefore, a need remains for a sustained release form of levodopa, or of carbidopa-levodopa combinations, that provides a constant delivery of active ingredient over a 12-24 hour period.

### SUMMARY OF THE INVENTION

The present invention provides a gastro-retentive dosage form of levodopa, and carbidopa-levodopa combinations, that provides a once-daily dosage. The invention relies on gastro-retentive technology (GRS) described herein to accomplish the retention of a dosage form in the stomach for a period of time of up to 6-24 hours or longer. Such a dosage form allows the prolonged, controlled delivery of levodopa to the upper small intestine, the optimum site for absorption of the drug, for up to 24 hours or more resulting in reduced frequency of dosing and a flat pharmacokinetic profile of levodopa. In addition, enhanced bioavailability provided by the prolonged retention of the dosage form at the site of optimal absorption, may result in reduced dosing requirement.

One aspect of the present invention thus provides a gastro-retentive dosage form of levodopa for oral administration to a patient in need thereof, said dosage form comprising (a) a tablet comprising a therapeutically effective amount of levodopa, a binder, and a pharmaceutically-acceptable gas-generating agent capable of releasing carbon dioxide upon contact with gastric fluid and (b) an expandable, hydrophilic, water-permeable and substantially gas-impermeable, membrane surrounding the tablet, wherein the membrane expands as a result of the release of carbon dioxide from the gas-generating agent upon contact with the gastric juice, whereby the dosage form becomes too large to pass into the patient's pyloric sphincter. Optionally, the dosage form is held within a covering that disintegrates without delay upon contact with the gastric fluid. In addition to levodopa, the tablet may contain carbidopa in amounts effective to provide enhanced availability of levodopa, as well as pharmaceutically acceptable excipients, diluents, glidants, lubricants, and the like.

Another aspect of the present invention provides a method of making a gastro-retentive dosage form of levodopa, wherein said method comprises (a) forming a tablet comprising levodopa, a binder and a pharmaceutically-acceptable gas-generating agent, (b) surrounding the tablet with an expandable, hydrophilic, water-permeable and substantially gas-impermeable, membrane, and (c) sealing the membrane to retard the escape of gas from within the sealed membrane. A further optional step comprises (d) encapulating the membrane-sealed tablet within a covering that disintegrates without delay upon contact with gastric fluid.

An additional aspect of the present invention provides a method of treating a patient in need thereof with a sustained release dosage of levodopa by orally administering to said patient a gastro-retentive dosage form of levodopa. In particular, patients suffering from Parkinson's disease will benefit from the method of treatment of the present invention.

### BRIEF DESCRIPTION OF THE DRAWINGS

FIG. 1 shows a flow diagram for the preparation of certain embodiments of the gastroretentive dosage forms of the present invention. The formulations contain Samarium Oxide, a radionucleotide, to allow measurement of the gastrointestinal transit of the dosage forms after administration to human volunteers, using scintigraphy.

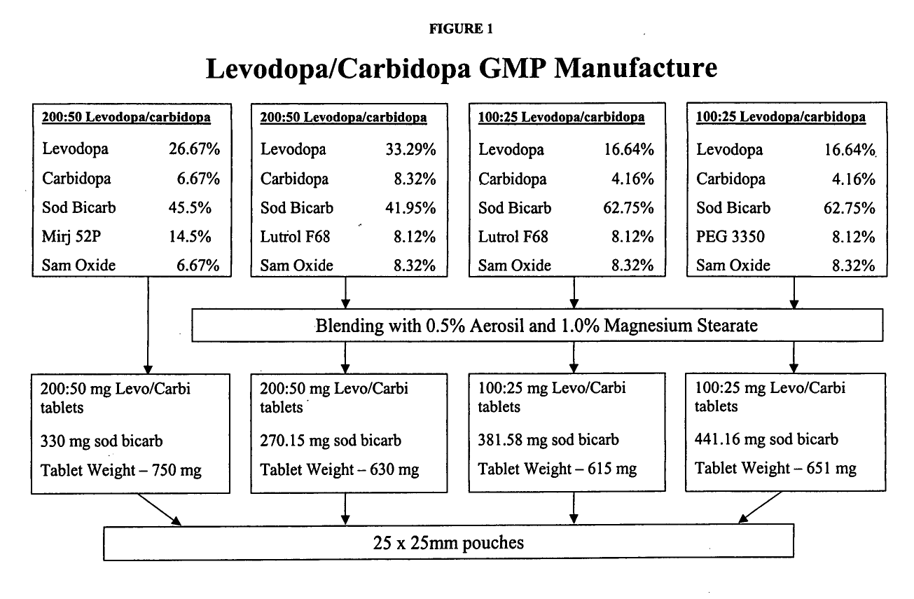

FIG. 2 is an in vitro dissolution profile for pouches containing tablets of Tablet Formulation 19 (Granule Formulation 9). The percentage of levodopa (black diamonds) or carbidopa (gray squares) released from the pouches in simulated gastric fluid as a function of time is shown. The results shown are for two separate dissolutions—one measured at time points between 0 and 8 hours and at 24 hours, and the other measured at time points between 10 and 34 hours. The measurements are plotted on the same curve.

FIG. 3 is an in vitro dissolution profile for pouches containing tablets of Tablet Formulation 20 (Granule Formulation 10). The percentage of levodopa (black diamonds) or carbidopa (gray squares) released from the pouches in simulated gastric fluid as a function of time is shown. The results shown are for two separate dissolutions—one measured at time points between 0 and 8 hours and at 24 hours, and the other measured at time points between 10 and 34 hours. The measurements are plotted on the same curve.

FIG. 4 is an in vitro dissolution profile for pouches containing tablets of Tablet Formulation 21 (Granule Formulation 11). The percentage of levodopa (black diamonds) or carbidopa (gray squares) released from the pouches in simulated gastric fluid as a function of time is shown. The results shown are for two separate dissolutions—one measured at time points between 0 and 8 hours and at 24 hours, and the other measured at time points between 10 and 34.5 hours. The measurements are plotted on the same curve.

FIG. 5 is a comparison of dissolution profiles showing time course of the release of levodopa for four different formulations of the dosage form: gray diamonds—tablet formulation 19; black triangle with solid line—tablet formulation 20; black triangle with dotted line—tablet formulation 21; gray square—granule formulation 12 (tablet).

FIG. 6 is a similar comparison profile of the release of carbidopa from four different dosage forms: Open circles—tablet formulation 19; open triangle with solid line—tablet formulation 20; X with dotted line—tablet formulation 21; gray square—granule formulation 12 (tablet).

FIG. 7 is a time course of the relative expansion of the pouches containing the tablet formulation, based on visual inspection in simulated gastric fluid on a scale of 0 to 3. A rating of 0 indicates the pouch is not inflated, 1 indicates beginning to inflate, 2 indicates almost inflated and 3 indicates fully inflated.

FIG. 8 is a time course of the expansion by volume of pouches containing tablets having Granule Formulation 12.

### DETAILED DESCRIPTION AND PRESENTLY PREFERRED EMBODIMENT

The present invention provides a gastro-retentive dosage form of levodopa for oral administration to a patient in need thereof, said dosage form comprising (a) a tablet comprising a therapeutically effective amount of levodopa, a binder, and a pharmaceutically-acceptable gas-generating agent capable of releasing carbon dioxide upon contact with gastric juice, and (b) an expandable, hydrophilic, water-permeable and substantially gas-impermeable, membrane surrounding the tablet, wherein the membrane expands as a result of the release of carbon dioxide from the gas-generating agent upon contact with the gastric juice causing the dosage form to become too large to pass into the patient's pyloric sphincter for a period of time. The gastro-retentive dosage form is optionally provided with a covering that disintegrates without delay upon contact with gastric fluid.

One embodiment of this invention can be seen as a gas generating inflatable system which is encapsulated in a hard gelatin dry-fill capsule. On contact with gastric fluid, the capsule dissolves to release a membrane pouch (e.g. about 25 mm×25 mm in size) which contains the active ingredient levodopa formulated with effervescent and rate controlling excipients. When water or gastric fluid penetrates the pouch, carbon dioxide is liberated from the tablet and this causes the pouch to inflate to a volume of up to about 20 mls. The gas-filled pouch is able to float on the aqueous phase and is retained in the stomach because it is too large to pass into the pyloric sphincter. The inflation of the pouch is a gradual process and carbon dioxide is released over a defined time period to maintain the inflation. Typically, the pouch remains inflated for a period of about 8-12 hours and can remain inflated for up to 24 hours or more. The period of inflation also reflects the gastric retention time. During its dwell time in the stomach, the levodopa and carbidopa present in the tablet component are released slowly into the surrounding body fluid, preferably by diffusion, through the membrane of the pouch. Since gastric juice is always being transported further, the active ingredient passes continuously and over a prolonged period into the duodenum, where it is absorbed over an extended period. The gastro-retentive form according to the invention therefore ensures continuous release of the levodopa and carbidopa in conjunction with uniform absorption. Once the gas generating formulation is depleted, the pouch deflates and flattens, becoming flexible enough to pass through the pylorus and hence empties from the stomach.

The gastro-retentive dosage form of the present invention is particularly advantageous for treating patients suffering from Parkinson's disease as it provides a sustained release of the active levodopa at a relatively constant level directly at the site of optimum absorption in the upper small intestine.

“Tablet” for the purposes of the present invention includes any solid pharmaceutical dosage form containing drug substances with or without suitable diluents, prepared by granulation, compression or molding methods, and also includes hard or soft gelatin capsules, granules, pills, and pellets.

The terms “gastric fluid” and “gastric juice” are used interchangeably throughout and refer to the endogenous fluid medium of the stomach, including water and secretions. “Simulated gastric fluid” means any fluid that is generally recognized as providing a useful substitute for authentic gastric fluid in experiments designed to assess the chemical or biological behavior of substances in the stomach. One such simulated gastric fluid is aqueous 0.1 N HCl, pH 1.2. It will be understood that the term “gastric fluid” or “gastric juice” used throughout the disclosure and claims means authentic (i.e. endogenous) gastric fluid or simulated gastric fluid.

The term “gastro-retentive form” denotes dosage forms which effect sustained release of the active ingredient in comparison with conventional dosage forms, such as customary tablets or capsules, while avoiding an undesirably high initial dose, the release being effected continuously over a relatively long period and controlled at a therapeutically effective level by prolonged retention of the dosage form in the stomach.

The tablet component of the gastro-retentive dosage form of the present invention comprises levodopa, as active ingredient, a binder and a pharmaceutically-acceptable gas-generating agent. The tablet component optionally comprises carbidopa in combination with the active ingredient levodopa. The tablet component may additionally contain suitable diluents, glidants, lubricants, acidulants, stabilizers, swelling agents and other pharmaceutically acceptable excipients.

Active

The active ingredient in the gastro-retentive dosage forms of the present invention is levodopa, which is variously known, inter alia, as L-dopa; β-(3,4-dihydroxyphenyl)-α-alanine; or 2-amino-3-(3,4-dihydroxyphenyl)propanoic acid. Levodopa can be used alone as the active ingredient or can be combined with carbidopa (S-α-hydrazino-3,4-dihydroxy-α-methylbenzenepropanoic acid monohydrate) in a weight ratio of levodopa to carbidopa of from about 20 to 1 to about 2 to 1, preferably from about 10 to 1 to about 2 to 1, most preferably from about 5 to 1 to about 3 to 1, in particular about 4 to 1. Levodopa is available commercially and its synthesis has been described in numerous publications, for example, Yamada et al., Chem. Pharm. Bull., 10: 693 (1962) and U.S. Pat. No. 4962223 and the references cited therein. Known therapeutic uses of levodopa include treatment of idiopathic and postencephalic parkinsonism, several extrapyramidal neuropathies, and depression. Carbidopa is commercially available. Levodopa-carbidopa combinations for the treatment of Parkinson's disease are well known and have been described, for example, in U.S. Pat. No. 4,900,755, and are commercially available (e.g. SINEMET®).

The tablet component contains the active ingredient levodopa in a therapeutically effective amount. The therapeutically effective amount per dose of levodopa for treatment of Parkinson's disease is between about 100 and about 400 mg /dosage. Typically, the levodopa is present in an amount from between 10% to about 50% of the total tablet weight, preferably between about 15% and about 40%. In general, this amount of levodopa will provide between about 100 mg and 250 mg of levodopa per dosage form, which amount is the preferred unit dosage range. Other therapeutically effective dosages can be readily determined by one of skill in the pharmaceutical or medical arts. Carbidopa, if present, will be included in accordance with the weight ratios discussed above. Typically, the carbidopa will be present at about 3% to about 8% of the total tablet weight.

Binder

The tablet component of the gastro-retentive dosage form comprises the active ingredient (that is, levodopa or combinations of levodopa and carbidopa), a gas-generating agent and a binder. Binders (also called wetting agents) are agents used to improve the cohesiveness of the tablet formulation, ensuring that the tablet will remain intact after formation. Suitable binders for use in the present invention include poloxamers, polyethylene glycols (e.g., PEG 3350), polyethylene glycol fatty acid esters (e.g., Myrj), glyceryl palmitostearate (e.g. Precirol AT05), polyoxyethylene alkyl ethers, glyceryl behenate (e.g., Compritol 888), stearoyl macrogol-32-glyceride (e.g., Gelucire), polyoxyethylene castor oil derivatives, polyoxyethylene sorbitan fatty acid derivatives, polyoxyethylene stearates, polyoxyethylene-polyoxypropylene copolymers (e.g. Lutrol or Pluronics), starches, gelatin, sugars such as lactose, sucrose, glucose and molasses, natural and synthetic gums such as acacia, sodium alginate, carboxymethylcellulose, methylcellulose, polyvinylpyrrolidone, ethyl cellulose and waxes. Especially preferred binders include Myrj52 (particularly Myrj52P or Myrj52FL), Lutrol F68, Compritol 888, Gelucire 50/13, PEG 3350, Precirol ATO5 methylcellulose and polyvinyl pyrrolidone.

The binder is present in the tablet component in an amount effective to provide cohesion to the final tablet form. The appropriate amount of binder can be readily determined by one of ordinary skill in the pharmaceutical arts and will depend, inter alia, upon the particular binder used and the method of preparation of the tablet. Typically, the binder is present in the tablet in an amount from between about 8% to about 15% of the total tablet weight.

Gas-generating agent

A gas-generating agent is included in the tablet component to generate the carbon dioxide gas that results in the expansion of the membrane component upon contact with gastric juice. Suitable gas-generating agents are, for example, solids that liberate this gas itself, for example under the action of body fluid or the hydrogen ions present therein. Such gas-generating agents are, for example, those capable of releasing carbon dioxide and include, but are not limited to, pharmaceutically acceptable mono- and di-basic salts of carbonic acid, for example alkali metal hydrogen carbonates or alkali metal carbonates, alkaline earth metal carbonates or ammonium carbonate.

Such mono- or di-basic salts of carbonic acid are especially sodium hydrogen carbonate (sodium bicarbonate) or sodium carbonate, potassium carbonate, calcium carbonate, magnesium carbonate, sodium glycine carbonate, or mixtures thereof. In order to increase the evolution of carbon dioxide, there may be added to the mentioned carbonates the acid component customarily used in effervescent mixtures, for example sodium dihydrogen phosphate or disodium hydrogen phosphate, sodium tartrate, sodium ascorbate or sodium citrate. Also suitable are yeasts which are likewise capable of generating carbon dioxide gas. When yeasts, for example baker's yeast, are used, the necessary nutrients, for example glucose, are added to the formulation. Preferably, in the present invention, the gas-generating agent will be sodium hydrogen carbonate.

The gas-generating agent will typically be present in the tablet component in an amount between about 30% and about 82% of the total tablet weight. Preferably, the gas-generating agent will be present at about 40% to about 82% of the total tablet weight.

Other Agents

In addition to the active ingredient, the binder and the gas-generating agent, the tablet component may also include one or more of diluents, glidants, lubricants, acidulants, swelling agents, surfactants and other pharmaceutically acceptable excipients. A diluent is a substance added to increase the bulk of a mixture to make a tablet a practical size for granulation, compression or molding when only a small amount of active is present. Suitable diluents include lactose, cellulose, dry starch, powdered sugar, dicalcium phosphate, calcium sulfate, sodium chloride, kaolin, mannitol, sorbitol, sucrose, inositol; preferred diluents are lactose, sorbitol, mannitol, cellulose and starch. A glidant (or flow-enhancing agent) is a substance that improves the flow characteristics of a powder mixture. Commonly used glidants include colloidal silicon dioxide, magnesium trisilicate, powdered cellulose, starch, tribasic calcium phosphate and talc. Glidants useful in this invention include these commonly used glidants and a preferred glidant is Aerosil 200, colloidal silicon dioxide. A lubricant is a substance that has a number of functions in the preparation of the tablet component of this invention, including preventing the adhesion of the tablet material to the surface of the dies and punches, reducing interparticle friction, facilitating the ejection of the tablet from the die cavity and in some instances, improving the rate of flow of the tablet granulation. Commonly used lubricants include talc, magnesium stearate, calcium stearate, zinc stearate, stearic acid, glyceryl monostearate, glyceryl palmitostearate, hydrogenated vegetable oils, hydrogenated castor oil, light mineral oil, sodium benzoate, sodium stearyl fumarate and polyethylene glycol (PEG). Any of the commonly used lubricants are suitable for use in the present invention. Preferably, magnesium stearate is used as a lubricant. An acidulant may be added to increase the release of carbon dioxide from this sodium hydrogen carbonate. Commonly used acidulants include citric acid, fumaric acid, malic acid and tartaric acid. It will be apparent from the foregoing that a single substance may serve more than one of the purposes described above.

Swelling Agents

In addition to the afore-mentioned gas-generating agents, it is also possible for intensifying the action of the agent to use pharmaceutically acceptable hydrophilic swelling agents, for example partially etherified cellulose derivatives, starches, water-soluble, aliphatic or cyclic poly-N-vinylamides, polyvinyl alcohols, polyacrylates, polymethacrylates, polyethylene glycols or mixtures of these auxiliaries. In certain embodiments, the hydrophilic swelling agent may also serve as a binder.

Hydrophilic, partially etherified cellulose derivatives are, for example, lower alkyl ethers of cellulose having an average degree of molar substitution (MS) of more than 1 and less than 3 and an average degree of polymerisation of approximately 100-5000.

The degree of substitution is a measure of the substitution of the hydroxy groups by lower alkoxy groups per glucose unit. The average degree of molar substitution (MS) is a mean value and indicates the number of lower alkoxy groups per glucose unit in the polymer.

The average degree of polymerisation (DP) is likewise a mean value and indicates the average number of glucose units in the cellulose polymer.

Lower alkyl ethers of cellulose are, for example, cellulose derivatives that are substituted at the hydroxymethyl group (primary hydroxy group) of the glucose unit forming the cellulose chains and optionally at the second and third secondary hydroxy group by C₁-C₄alkyl groups, especially methyl or ethyl, or by substituted C₁-C₄alkyl groups, for example 2-hydroxyethyl, 3-hydroxy-n-propyl, carboxymethyl or 2-carboxyethyl.

Suitable lower alkyl ethers of cellulose are especially methylcellulose, ethylcellulose, methylhydroxyethylcellulose, methylhydroxypropylcellulose, ethylhydroxyethylcellulose, hydroxyethylcellulose, hydroxypropylcellulose, carboxymethylcellulose (in salt form, for example sodium salt form) or methylcarboxymethylcellulose (likewise in salt form, for example sodium salt form).

A starch suitable for use as hydrophilic swelling agent is, for example, a mixture of approximately 15-20% amylose (molar mass approximately 50,000 to 200,000) and 80-85% amylopectin (molar mass approximately 100,000 to 1,000,000), for example rice, wheat or potato starch, and also starch derivatives, such as partially synthetic amylopectin, for example sodium carboxymethylamylopectin, and alginates of the alginic acid type.

Water-soluble, aliphatic or cyclic poly-N-vinylamides are, for example, poly-N-vinyl-methylacetamide, poly-N-vinylethylacetamide, poly-N-vinylmethylpropionamide, poly-N-vinylethylpropionamide, poly-N-vinylmethylisobutyramide, poly-N-vinyl-2-pyrrolidone, poly-N-vinyl-2-piperidone, poly-N-vinyl-ε-caprolactam, poly-N-vinyl-5-methyl-2-pyrrolidone or poly-N-vinyl-3-methyl-2-pyrrolidone, especially poly-N-vinylpyrrolidone having a mean molar mass of approximately 10,000-360,000, for example the polyvinylpyrrolidone obtainable under the trade mark Kollidon® (BASF).

Suitable polyvinyl alcohols have a mean molar mass of approximately 15,000 to 250,000 and a degree of hydrolysis of approximately 70-99%. Preferred polyvinyl alcohols are those having a degree of hydrolysis of approximately 70-88% (partially hydrolysed polyvinyl alcohol), for example the polyvinyl alcohol obtainable under the trade name Mowiol® (Hoechst) denoted by MOWIOL 3-83, 4-80, 4-88, 5-88 or 8-88.

Hydrophilic polyacrylates that can be used as swelling agents have a mean molecular weight of approximately 8.6×10⁵ to 1.0×10⁶. The polyacrylic acid chains carry a greater or smaller number of short side chains and so the individual commercial forms differ in this respect, as well as in having different molecular weights. Neutralised (for example with dilute aqueous sodium hydroxide solution) polyacrylic acid derivatives of the commercial form Carbopol® (Goodrich), for example CARBOPOL 934 P or CARBOPOL 940, are preferred.

Suitable polymethacrylates are likewise swellable and have a mean molecular weight of more than 1.0×10⁶. Preferred commercial forms that can be used are the polymers of methacrylic acid and methacrylic acid esters of the Eudragit® type, for example EUDRA-GIT L or EUDRAGIT S (Röhm GmbH).

Suitable polyethylene glycols have an average molecular weight of approximately 4000 to 6000. Pharmaceutical-quality commercial forms are preferred, for example polyethylene glycol such as Lutrol® (BASF), Polydiol®, Polywachs® Hüls), Polyglykol®, Lanogen® (Hoechst), Carbowax® (Union Carbide), Plurocol® (Wyandotte) or Tetronic® (Kuhlmann).

Suitable hydrophilic swelling agents are also homopolymers, such as polyhydroxyalkyl methacrylate having a molecular weight from 5,000 to 5,000,000 anionic or cationic hydrogels, mixtures of agar and carboxymethylcellulose, swellable agents consisting of methylcellulose in admixture with weakly cross-linked agar, or water-swellable polymers that can be produced by dispersion of a finely particulate copolymer of maleic acid anhydride and styrene, or tragacanth, gelatine or swellable ion exchange resins.

Swellable ion exchangers are, for example, copolymer resins having acidic groups, for example, sulfonic acid groups or salt forms thereof based on styrene-divinylbenzene. Such copolymer resins consist of cross-linked styrene polymers which are obtained by copolymerization of styrene with divinylbenzene as cross-linking agent. Customary derivisation reactions, for example sulfonation reactions, are used to incorporate acidic groups, such as sulfo groups, into the structure. The preparation and the properties of these resins are known. Reference is made to the article in Ullmanns Enzyklopädie der Technischen Chemie, 4^{th} Edition, Vol. 13, pp. 279 ff., and to Kirk-Othmer, Encyclopaedia of Chemical Technology, J. Wiley, Vol. 13, pp. 678 ff., and to the numerous literature references cited therein.

Preferred ion exchange resins are those having quaternary ammonium groups or sulfonic acid groups based on styrenedivinylbenzene which are commercially available and are acceptable for use in pharmaceutical formulations, for example resins marketed by the firm Rohm and Haas under the trade mark Amberlite® IRP-69.

Surfactants

The tablet component can also contain the customary pharmaceutical formulation adjuncts that are used at present for the manufacture of oral dosage forms, such as tablets, for example surface-active substances, for example so-called surfactants, for example anionic surfactants of the alkyl sulfate type, for example sodium, potassium or magnesium n-dodecyl sulfate, n-tetradecyl sulfate, n-hexadecyl sulfate or n-octadecyl sulfate, alkyl ether sulfate, for example sodium, potassium or magnesium n-dodecyloxyethyl sulfate, n-tetradecyloxyethyl sulfate, n-hexadecyloxyethyl sulfate or n-octadecyloxyethyl sulfate, or alkanesulfonate, for example sodium, potassium or magnesium n-dodecanesulfonate, n-tetradecanesulfonate, n-hexadecanesulfonate or n-octadecanesulfonate.

Suitable surfactants are also nonionic surfactants of the fatty acid/polyhydroxy alcohol ester type, such as orbitan monolaurate, monooleate, monostearate or monopalmitate, sorbitan tristearate or trioleate, polyoxyethylene adducts of fatty acid/polyhydroxy alcohol esters, such as polyoxyethylene sorbitan monolaurate, monooleate, monostearate, monopalmitate, tristearate or trioleate, polyethylene glycol/fatty acid esters, such as polyoxyethylene stearate, polyethylene glycol 400 stearate or polyethylene glycol 2000 stearate, especially ethylene oxide/propylene oxide block copolymers of the Pluronics® (BWC) or Synperonic® (ICI) type, myristates and their condensation products, or ethylene oxide homopolymers having a degree of polymerisation of approximately 2,000 to 100,000, which are known, for example, under the trade name Polyox® (Union Carbide).

Tablet Preparation

The tablet component can be formed by any conventional tabletting method, such as, mixing, dry granulation, wet granulation, melt granulation, fluid bed granulation, direct compression, molding, or extrusion. Preferably, the tablet component is prepared using fluid bed granulation, melt granulation or direct compression methods.

Typically, for preparation of tablet for use in the gastro-retentive dosage form of the present invention, the appropriate amounts of levodopa, carbidopa (if applicable), a gas-generating agent (typically sodium bicarbonate) and a binder are mixed in a high shear mixer for a short period of time. The mixture is heated to a temperature that is approximately 5°-10° C. above the melting temperature of the binder. The heated mixture is then blended and granulated and milled to obtain particles of fairly homogenous size (approximately 1.0 mm or less). The granules are mixed with a glidant (preferably Aerosil 200) and a lubricant (preferably magnesium stearate). This mixture is formed into tablets by conventional tabletting methodology.

Alternatively, the gas-generating agent and a diluent such as sorbitol/mannitol are mixed in a fluid bed machine and a binder solution is sprayed on the mixture. The material is dried by heating, cooled and blended with the levodopa, carbidopa (if applicable), glidant and lubricant. Tablets are formed from the granules using conventional tabletting methods.

The tablet can be of any convenient shape and size suitable for oral administration and/or for ease of preparation of the gastro-retentive dosage form. Typically, the tablet will be round, flat, bevel-edged or oval and typically will be approximately 5 mm shorter than the longest internal dimension of the pouch. Generally, the tablet will be no more than 20 mm in its longest dimension. The total tablet weight will generally vary from about 250 mg to about 2500 mg, preferably will vary from about 500 mg to about 1000 mg, more preferably between about 600 mg and about 800 mg, before addition of the membrane pouch and the optional covering.

Expandable Membrane

The hydrophilic membrane, which is expandable at the site of use and is permeable to body fluid, consists of a plastic or wax-like, pharmaceutically acceptable polymeric material that is substantially gas-impermeable to the gas generated by the gas-generating agent. By “substantially gas-impermeable” is meant that the flow of gas through the membrane is impeded sufficiently to allow expansion of the membrane sachet or pouch upon the generation of gas from the gas-generating agent contained in the tablet component for a suitable period of time. Because of its hydrophilic properties, the membrane can absorb body fluid, such as gastric fluid, and can effect retarded and continuous release of controlled amounts of the levodopa contained in the tablet component by means of diffusion or optionally by the use of osmosis.

Suitable plastic or wax-like polymeric materials for the expandable hydrophilic membrane include for example hydrophilic foils, for example foils of cellulose ethers, such as methyl- or ethyl-cellulose, hydroxypropylcellulose, methyl- or ethyl-hydroxyethylcellulose, methyl- or ethyl-hydroxypropylcellulose carboxymethylcellulose, polyvinyl alcohol, polyvinyl acetate, polyvinylpyrrolidone, polyacrylonitrile, mixtures of polyvinylpyrrolidone with polyvinyl alcohol, resins based on phthalic acid anhydride/polyhydroxy alcohol, urethanes, polyamides, shellac, etc.

Especially preferred are polyvinyl alcohols having a degree of hydrolysis of more than 92% (fully hydrolysed polyvinyl alcohol), especially more than 97%, for example MOWIOL of the 98 series, for example MOWIOL 4-98, 10-98, 20-98, 28-99, 56-98 and 66-100, PVAU228-08. Particularly preferred are MOWIOL 28-99 and PVAU228-08.

To these materials it is possible to add further adjuncts, for example plasticisers, which improve the elasticity of the membrane, for example glycerol, polyethylene glycol/fatty acid esters, such as polyethylene glycol 400 stearate or polyethylene glycol 2000 stearate, triethyl citrate, diethyl phthalate, diethyl sebacate, and the like. The amount of plasticiser added is approximately from 0.01 to 60% by weight, based on the total weight of the dosage form. Preferably, glycerol at 10-30% w/w is used as the plasticizer, most preferably 20%.

In one embodiment, the expandable membrane is produced by preparing a homogeneous mixture of polyvinyl alcohol and additives, such as plasticisers, for example glycerol and/or polyethylene glycol 400 stearate, by dissolution in water, which is optionally heated, and evaporation to form layers of suitable thickness, for example 100 mm, or by allowing a solution of polyvinyl alcohol in water (without additives) to evaporate. The film or the foil which is obtainable after evaporation of an aqueous solution of polyvinyl alcohol, especially polyvinyl alcohol having a degree of hydrolysis of more than 97%, and polyethylene glycol/fatty acid ester, for example polyethylene glycol 400 stearate or polyethylene glycol 2000 stearate, optionally with the addition of plasticisers, such as glycerol, is distinguished by a high degree of extensibility. A film-like residue which can be obtained after evaporation of an aqueous solution containing approximately 40-85% polyvinyl alcohol, 0-40% polyethylene glycol stearate and 10-30% glycerol has particularly advantageous properties. This film is distinguished by particularly good extensibility. This film can be easily cut and formed into pouches or sachets to accommodate individual tablet components or used as a sheet to fold around the tablet component or several sheets of membrane film can be used to sandwich the tablet components.

Optional Covering

In certain embodiments, the gastro-retentive form according to the invention can be provided with a covering which surrounds or contains the tablet component and the membrane component and which disintegrates without delay under the action of body fluid at the site of use and which consists of a film coating or, preferably, a covering in capsule form.

Suitable film coatings delay the release of active ingredient only slightly or not at all. Water-soluble film coatings from approximately 20 μm to approximately 150 μm in thickness are preferred. Suitable film coating materials are especially hydrophilic cellulose derivatives, such as cellulose ethers, for example methylcellulose, hydroxypropylcellulose or especially hydroxypropylmethylcellulose, mixtures of polyvinylpyrrolidone or of a copolymer of polyvinylpyrrolidone and polyvinyl acetate with hydroxypropylmethylcellulose, mixtures of shellac with hydroxypropylmethylcellulose, polyvinyl acetate or copolymers thereof with polyvinylpyrrolidone, or mixtures of water-soluble cellulose derivatives, such as hydroxypropylmethylcellulose, and water-insoluble ethylcellulose. These coating agents can, if desired, be used in admixture with other adjuncts, such as talc, wetting agents, for example polysorbates (for example to facilitate application), or pigments (for example for identification purposes). Depending upon the solubility of the components, these coatings are applied in aqueous solution or in organic solution (for example solutions of shellac or ethylcellulose in organic solvents). It is also possible to use mixtures of acrylates that are water-insoluble per se, for example the copolymer of ethyl acrylate and methyl methacrylate, which are used in aqueous dispersion, with water-soluble adjuncts, for example lactose, polyvinylpyrrolidone, polyethylene glycol or hydroxypropylmethylcellulose.

Instead of using a film-like coating, the gastro-retentive forms according to the invention can be provided with a covering in capsule form. Hard gelatin capsules having high watersolubility and/or swellability are preferred. Size 000, Size 00 and Size 0 dry-fill capsules are preferred, in order to accommodate the membrane enclosed tablets.

When present, the covering is preferably a dry-fill capsule, more preferably a hard gelatin dry-fill capsule.

Preparation of the Gastro-retentive Forms

In one aspect, the present invention provides a method of making a gastro-retentive dosage form of levodopa, which method comprises: forming a tablet comprising levodopa, a binder and a pharmaceutically-acceptable gas-generating agent, surrounding the tablet with an expandable, hydrophilic, water-permeable and substantially gas-impermeable membrane, and sealing the membrane to retard the escape of gas from within the sealed membrane. Optionally, the method comprises the additional step of encapsulating the sealed membrane within a covering that disintegrates without delay upon contact with gastric fluid.

As described above, the tablet component can be formed using any convenient tabletting method. Such methods are well known in the art and are described, for example, in Remington: the Science and Practice of Pharmacy 19^{th} Ed. 1995 Mack Publishing Co. Easton Pa.

In the gastro-retentive dosage form of the present invention, the tablet component will be surrounded by the expandable membrane component. The membrane surrounds the tablet on all sides and is sealed to retard the escape of gas generated by the gas-generating agent contained in the tablet. This surrounding can be accomplished in various ways. The membrane may be a preformed sachet or pouch that contains an opening large enough for insertion of the tablet component. After insertion of the tablet, the opening is sealed by appropriate means, for example heat and/or pressure. Alternatively, the membrane may be formed around the tablet, for example as a coating on the tablet that completely surrounds the tablet, or may be formed by sandwiching the tablet component between two or more separate layers of membrane material, or one membrane layer folded over the tablet, and sealing the membrane layers together around the tablet by heat and/or pressure. Typically, the membrane pouch surrounding the tablet component will be as small as possible consistent with the need to accommodate the tablet component and provide for sufficient expansion of the dosage form in the stomach.

As mentioned, the hydrophilic membrane is typically prepared in the form of a sachet or pouch into which the tablet component can be inserted. Such a pouch or sachet is readily prepared from the membrane film prepared as described herein. After insertion of the tablet, the pouch can be sealed around the tablet to retard the escape of gas generated by the gas-generating agent in the tablet component. The sachet or pouch can be any convenient shape, typically will be rectangular or circular. Typically, the uninflated membrane sachet or pouch is about 20-25 mm in the longest dimension and may be shorter, depending on the size of the tablet component that must be accommodated. In some embodiments, the membrane film will not be preformed into pouches but will be used as a film layer to surround the tablet component, either by sandwiching the tablet between two (or more) membrane layers or by folding a single layer over the tablet. The membrane layers will be sealed on all sides surrounding the tablet and cut along the seal to produce the dosage form. Multiple dosage forms may be produced simultaneously in this way by using a membrane layer large enough to accommodate multiple tablets, sealing the membrane layers between the tablets and cutting at the sealed membrane to produce the dosage forms.

It is also possible for the tablet component to be surrounded not by one but by several coverings of expansible permeable material. With such a multi-layered arrangement, it is also possible for a formulation of the levodopa, or constituents of the formulation, for example the gas-generating agent, such as sodium hydrogen carbonate, to be located between the individual layers. With a multi-layered arrangement it is possible to achieve an even longer dwell time of the dosage form at the site of action, for example in the stomach. In addition, the expansible membrane (b) may itself, contain physiologically active substances.

In a preferred form of the process, the expandable membrane surrounding tablet component is produced first, for example by preparing a homogeneous mixture of polyvinyl alcohol and additives, such as plasticisers, for example glycerol and/or polyethylene glycol 400 stearate, by dissolution in water, which is optionally heated, and evaporation to form layers of suitable thickness, for example 100 mm, or by allowing a solution of polyvinyl alcohol in water (without additives) to evaporate. The layers are cut into strips of a suitable size and the active ingredient formulation consisting of the tablet component is applied. This can be effected for example, by filling the still open sachet, which is then closed completely, for example by sealing, for example with heat and/or pressure. The sealed sachets can then be filled into dry-fill capsules.

The gastro-retentive dosage form according to the invention can be of various shapes and may be, for example, round, oval, oblong, tubular and so on, and may be of various sizes depending upon the size and shape of the tablet component. In addition, the dosage form may be transparent, colourless or coloured in order to impart to the product an individual appearance and the ability to be immediately recognised.

In some embodiments, the gastro-retentive dosage form can be prepared using microparticulates or nanoparticulates comprising the active (i.e., levodopa or levodopa:carbidopa combinations) in lieu of a tablet. The microparticulates or nanoparticulates will comprise levodopa, a binder and a gas-generating agent, optionally carbidopa, and other optional components as described for the tablets. The microparticulates or nanoparticulates are prepared using, for example, the granulation techniques described herein or other well known methods for preparing microparticulates and nanoparticulates.

Particularly preferred gastro-retentive forms and methods of making the same are similar to those described in U.S. Pat. No. 4,996,058, which is incorporated herein by reference in its entirety.

Method of Treatment

The present invention provides a method of treating a patient suffering from Parkinson's disease by orally administering to the patient the gastro-retentive levodopa dosage form. The gastro-retentive form according to the invention is suitable for oral administration. The prolonged dwell time in the stomach of the gastro-retentive form of the present invention provides for prolonged sustained release of the levodopa or levodopa-carbidopa combinations at the site of optimum absorption for the levodopa. The sustained release of the active ingredient provided by the gastro-retentive form reduces the need for frequent dosing. Thus, the gastro-retentive dosage forms of the present invention are typically administered once or twice in a 24 hour period, preferably once in 24 hours, but may be administered more or less frequently depending on the requirement of the patent.

Article of Manufacture

In another aspect the present invention provides an article of manufacture comprising a the gastro-retentive levodopa dosage form, packaging material containing the dosage form and optionally a label or insert containing instructions for use of the dosage form for treatment of Parkinsons disease. The dosage form provided in the article of manufacture preferably includes a hard gelatin dry-fill capsule covering. The article of manufacture preferably comprises a dosage form comprising carbidopa in combination with levodopa at weight ratios described elsewhere herein. Combinations of 100 mg levodopa:25 mg carbidopa per dosage form and 200 mg levodopa:50 mg carbidopa per dosage form are more preferred. Individual dosage forms may be packaged separately into single containers or the packaging material may be provided with a plurality of dosage forms.

The following examples are provided for illustrative purposes and are not intended to limit the invention in any way.

### EXAMPLE 1

Preparation of Granules

Levodopa, carbidopa (if applicable to the batch), sodium bicarbonate and a binder (300 g batch size) were mixed at 200 rpm in the Rotolab high shear mixer for 5 minutes. The heating jacket was switched on and heated until the desired product temperature was reached. The desired temperature was 5°-10° above the melting temperature of the binder used. The materials were blended and granulated for a further 15 minutes. The molten material was then removed from the granulation bowl and milled using the Comill, initially using a screen mesh of 1.7 mm and finally a 0.7 mm screen mesh. Dose strengths evaluated were: 100 mg Levodopa, 100:25 mg Levodopa: Carbidopa, 200 mg Levodopa and 200:50 mg Levodopa:Carbidopa. A summary of the granules made is shown in Table 1.

Preparation of Tablets

The Levodopa or Levodopa:Carbidopa granules were placed in a bag and Aerosil 200 was sieved into the bag containing the granules using a lmm mesh. Bag blending occurred for 3 minutes. Once complete, magnesium stearate was sieved into the above bag through a 0.5 mm mesh and bag blended for a further 3 minutes. Tabletting was carried out using a Piccola 10 station using a 16×6mm flat beveled punch. A summary of the tablets made is shown in Table 2.

TABLE 1
Summary of granule compositions

| Granule | |
|---|---|
| Formulation | |
| No.: | Granule composition |
| 1 | 8.1% binder (e.g. either Myrj 52 FL, Lutrol F68, PEG 3350 or Precirol ATO 5), 76.8% |
| | sodium bicarbonate and 15.6% levodopa yielding potency of 156 mg/g Levodopa |
| | granules |
| 2 | 8.1% binder (e.g. either Myrj 52 FL, Lutrol F68 or PEG 3350), 72.4% sodium |
| | bicarbonate, 15.6% levodopa and 3.9% carbidopa yielding potency of 156:39 mg/g |
| | Levodopa:Carbidopa granules |
| 3 | 8.1% binder (e.g. either Myrj 52 FL, Lutrol F68, PEG 3350 or Precirol ATO 5), 62.9% |
| | sodium bicarbonate and 29.0% levodopa yielding potency of 290 mg/g Levodopa |
| | granules |
| 4 | 8.1% binder (e.g. either Myrj 52 FL, Lutrol F68 or PEG 3350), 55.6% sodium |
| | bicarbonate, 29.0% levodopa and 7.3% carbidopa yielding potency of 290:73 mg/g |
| | Levodopa:Carbidopa granules |

TABLE 2
Summary of tablet compositions manufactured

| Tablet | |
|---|---|
| Formulation | |
| No.: | Tablet composition |
| 1 | 100 mg Levodopa tablet, target tablet weight = 650 mg consisting of 52 mg binder (either |
| | Myrj 52FL, Lutrol F68 or PEG 3350), 488.25 mg sodium bicarbonate, 100 mg |
| | Levodopa, 3.25 mg Aerosil 200 and 6.50 mg magnesium stearate |
| 2 | 100:25 mg Levodopa:Carbidopa tablet, target tablet weight = 650 mg consisting of 52 mg |
| | binder (either Myrj 52FL, Lutrol F68 or PEG 3350), 463.25 mg sodium bicarbonate, |
| | 100 mg Levodopa, 25 mg Carbidopa, 3.25 mg Aerosil 200 and 6.50 mg magnesium |
| | stearate |
| 3 | 200 mg Levodopa tablet, target tablet weight = 700 mg consisting of 56 mg binder (either |
| | Myrj 52FL, Lutrol F68 or PEG 3350), 433.25 mg sodium bicarbonate, 200 mg |
| | Levodopa, 3.5 mg Aerosil 200 and 7.0 mg magnesium stearate |
| 4 | 200:50 mg Levodopa:Carbidopa tablet, target tablet weight = 700 mg consisting of 56 mg |
| | binder (either Myrj 52FL, Lutrol F68 or PEG 3350), 383.25 mg sodium bicarbonate, |
| | 200 mg Levodopa, 50 mg Carbidopa, 3.5 mg Aerosil 200 and 7.0 mg magnesium stearate |

### EXAMPLE 2

Fluid Bed Granulation

Sodium Bicarbonate, Sorbitol (or Sorbitol:Mannitol 60:40 ratio) were mixed in the fluid bed machine (Niro Aeromatic, Strea 1). A binder solution of PEG 6000 and water (16% w/w), PVP K25 and water (16% w/w), or water alone was sprayed onto the bicarbonate mixtures. The materials were dried for a further 30 minutes at 50° C. and then cooled for a further 30 minutes. A sample of the granule was taken and analyzed visually by light microscopy and tested for moisture content. The granules were blended with levodopa and carbidopa, magnesium stearate and Aerosil 200 and tabletted on the Single Station Fette Tabletting Machine using a 12mm round punch. A summary of the granules and tablets prepared is shown in Tables 3 and 4, respectively.

TABLE 3
Summary of granule compositions

| Granule | |
|---|---|
| Formulation | |
| No.: | Granule composition |
| 5 | 84.7% sodium bicarbonate, 12.7% sorbitol and |
| | 2.6% PVP K25 |
| 6 | 84.7% sodium bicarbonate, 7.6% sorbitol, |
| | 5.1% mannitol and 2.6% PVP K25 |
| 7 | 74.9% sodium bicarbonate, 22.3% sorbitol and |
| | 2.8% PVP K25 |
| 8 | 74.9% sodium bicarbonate, 22.3% sorbitol and |
| | 2.8% PEG 6000 |

TABLE 4
Summary of tablet compositions

| Tablet | |
|---|---|
| Formulation | |
| No.: | Tablet composition |
| 5 | 100 mg Levodopa tablet, tablet consisting of 400 mg sodium bicarbonate, 60 mg |
| | sorbitol, 12 mg PVP K25, 100 mg Levodopa, 3 mg Aerosil 200 and 6 mg magnesium |
| | stearate. |
| 8 | 200 mg Levodopa tablet, tablet consisting of 400 mg sodium bicarbonate, 60 mg |
| | sorbitol, 12 mg PVP K25, 200 mg Levodopa, 3 mg Aerosil 200 and 6 mg magnesium |
| | stearate. |
| 11 | 200 mg Levodopa tablet, tablet consisting of 400 mg sodium bicarbonate, 60 mg |
| | sorbitol, 12 mg PEG 6000, 200 mg Levodopa, 3 mg Aerosil 200 and 6 mg magnesium |
| | stearate. |
| 12 | 100:25 mg Levodopa:Carbidopa tablet, consisting of 400 mg sodium bicarbonate, 60 mg |
| | sorbitol, 12 mg PVP K25, 100 mg Levodopa, 25 mg Carbidopa 3 mg Aerosil 200 and |
| | 6 mg magnesium stearate. |
| 15 | 200:50 mg Levodopa:Carbidopa tablet, tablet consisting of 400 mg sodium bicarbonate, |
| | 60 mg sorbitol, 12 mg PVP K25, 200 mg Levodopa, 50 mg carbidopa, 3 mg Aerosil 200 |
| | and 6 mg magnesium stearate. |
| 16 | 200:50 mg Levodopa:Carbidopa tablet, tablet consisting of 400 mg sodium bicarbonate, |
| | 36 mg sorbitol, 24 mg mannitol, 12 mg PVP K25, 200 mg Levodopa, 50 mg carbidopa, |
| | 3 mg Aerosil 200 and 6 mg magnesium stearate. |
| 18 | 200:50 mg Levodopa:Carbidopa tablet, tablet consisting of 400 mg sodium bicarbonate, |
| | 60 mg sorbitol, 12 mg PEG 6000, 200 mg Levodopa, 50 mg Carbidopa, 3 mg Aerosil 200 |
| | and 6 mg magnesium stearate. |

Example 3-Preparation of Levodopa:Carbidopa granules (Lutrol F68 and PEG 3350 Prototype) for Biological Testing

Levodopa, carbidopa, sodium bicarbonate, samarium oxide (SmO₃) and either Lutrol F68 or PEG 3350 (300 g batch size) were weighed out and mixed at 200 rpm in the Rotolab for 5 minutes in the proportions set forth in Table 5. The samarium oxide was used in these preparations as a radioactive tracer for analysis of the gastrointestinal transit of the dosage form. The heating jacket was switched on. When the desired product temperature was reached, the materials were blended and granulated for a further 15 minutes. The molten material was then removed from the granulation bowl and milled using the Comill, initially using a screen mesh of 1.7 mm and finally a 0.7 mm screen mesh. Dose strengths manufactured were: 100:25 mg Levodopa:Carbidopa and 200:50 mg Levodopa:Carbidopa.

The Levodopa:Carbidopa granules were bag blended by hand in several batches. Equal quantities of the granules manufactured from each batch were placed into a polythene bag of adequate size after weighing and blended for 15 minutes by gentle shaking. Once completed, samples of the granule blends were analysed.

TABLE 5

| Granule | Granule | Granule |
|---|---|---|
| Formulation 9 | Formulation 10 | Formulation 11 |
| 8.1% PEG 3350 | 8.1% Lutrol F68 | 8.1% Lutrol F68 |
| 62.8% NaHCO₃ | 62.8% NaHCO₃ | 42.0% NaHCO₃ |
| 8.3% SmO₃ | 8.3% SmO₃ | 8.3% SmO₃ |
| 16.6% Levodopa | 16.6% Levodopa | 33.3% Levodopa |
| 4.2% Carbidopa | 4.2% Carbidopa | 8.3% Carbidopa |
| 166:42 mg/g | 166:42 mg/g | 333:83 mg/g |
| (levodopa:carbidopa | (levodopa:carbidopa | (levodopa:carbidopa |
| mg/g) | mg/g) | mg/g) |

The required weight of the Levodopa:Carbidopa granule blends was placed in a bag and Aerosil 200 was sieved into the bag containing the granules using a lmm mesh. Bag blending was then carried out for 3 minutes. Once complete, magnesium stearate was sieved into the above bag through a 0.5 mm mesh and bag blended for a further 3 minutes. Tabletting was carried out using a Piccola 10 station using a 16×6mm flat bevelled punch. The tablets contained 98.5% blended granules from granule formulations 9, 10 or 11, 0.5% Aerosil 200 and 1.0% magnesium stearate, producing tablets having tablet formulations 19, 20 or 21, respectively.

A pouch manufacturing machine was used to seal the tablet in the film pouches. Two rolls of PVA film, produced as described in Example 5, were fed into the machine (upper and lower roll). The levodopa or carbidopa/levodopa tablet was placed on the lower film roll and the upper film roll was fed over the tablet, vacuum pulled heat applied and a sealed pouch was formed. The pouch was then cut from the film, rolled or folded and filled into a gelatin capsule. Sealed pouches (25×25mm) were obtained by cutting them out with scissors.

Example 4- Preparation of Levodopa: Carbidopa granules (MYRJ 52P prototype)

A water bath was warmed to 60° C. The required amounts of levodopa, carbidopa, sodium bicarbonate and samarium oxide as set forth in Table 6 were weighed out into separate containers. The Myrj 52 was weighed into a stainless steel container. Once completed, the levodopa, carbidopa, samarium oxide and sodium bicarbonate were added to the stainless steel container containing the Myrj 52 and mixed for 10 minutes at room temperature. The stainless steel container was then placed into the water bath (temperature taken with a thermometer) and mixed for a further 20 minutes to melt the Myrj 52. Once completed, the granules were cooled and sieved through a 2.0 mm mesh sieve followed by a 1.0 mm mesh sieve into polyethylene bags respectively.

TABLE 6
Granule Formulation 12
(% w/w) composition

```
14.5% Myrj52
45.5% NaHCO₃
  6.7% SmO₃
26.7% Levodopa
  6.7% Carbidopa
267:67 mg/g
```

Levodopa:Carbidopa tablets were prepared using the Enerpac Single Station Press with a 16×12 mm flat rectangle punches. The compression force used was 100 bar. Briefly, 750 mg of the Levodopa:Carbidopa granules were weighed out and transferred to the die. The granules were then compressed into tablets one at a time. The tablets were removed from the punch and placed onto a stainless steel tray for storing with dessicant prior to pouching.

PVA pouches containing the tablets were prepared as described in Example 3.

Example 5-Preparation of PVA Expandable Membrane

Appropriate amounts of glycerol and USP water are mixed in a mixing drum and PVA is added. The mixture is deaerated for 20 minutes and heated gradually, with increased mixing over 5-6 hours to a temperature of 95° C. The mixture is allowed to cool slowly for a period of about eight hours.

The PVA film is made by coating the solution onto a PET web. The web is then passed through an oven at temperatures above 100° C. to allow the PVA solution to dry into a film. After drying, the film is rewound to a master roll, which is then cut to the required size and sealed in aluminum foil bags with dessicant.

Using the above procedure, a 150 μm thick membrane is produced from 20% glycerol and 80% PVA (MOWIOL 28-99) and used to prepare dosage forms as described in Example 3.

Example 6-Simulated Gastric Release

The release of levodopa and carbidopa from unencapsulated pouches in a simulated gastric fluid (USP II-Mesh, Dissolution Medium—pH 1.2, 0.1N HCl) was measured over time. The pouches were placed in 900 mls at 37.5° C. with stirring at 50 rpm for up to 40 hours. Samples of 3 mls were removed by syringe at various time points for analysis. The samples were filtered through a 0.45 μm Millipore Millex HV Hydrophilic PVDF filter and the amount of levodopa or carbidopa was determined by HPLC with UV detector at a wavelength of 280 nm.

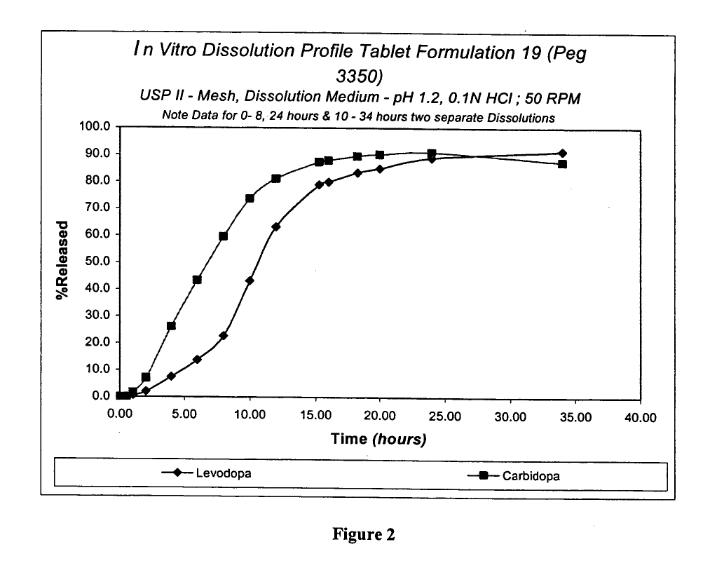

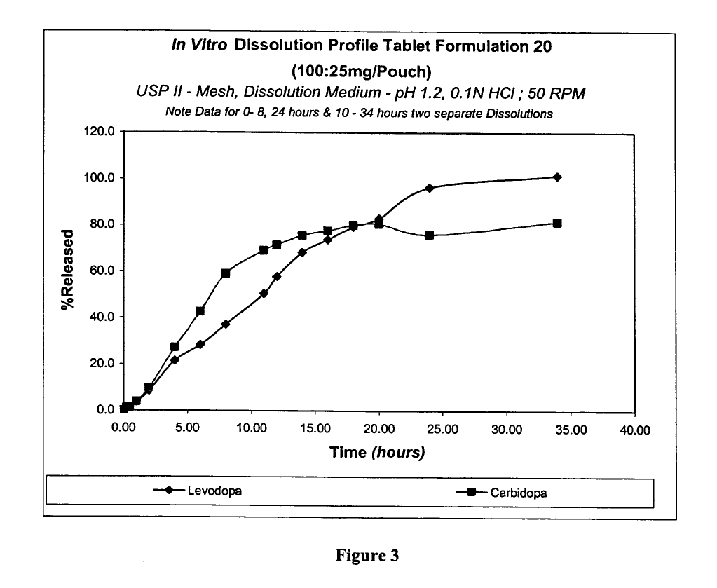

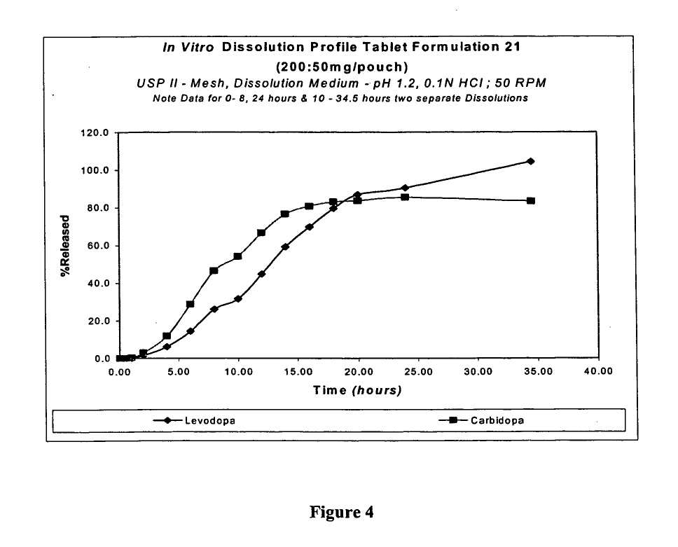

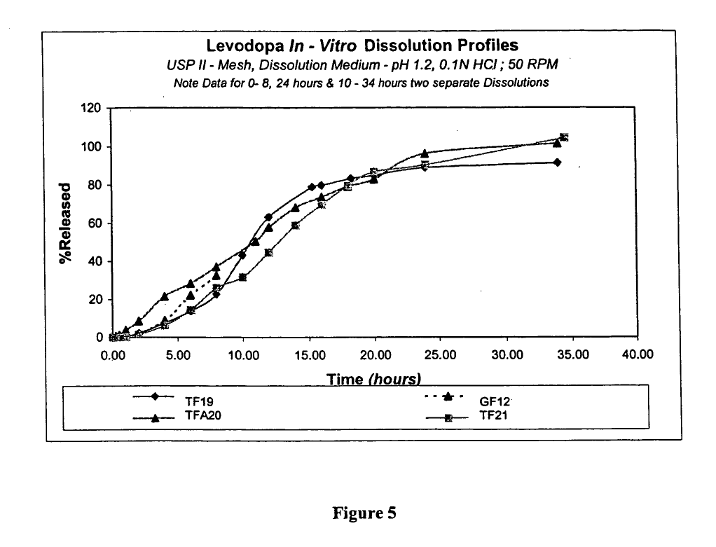

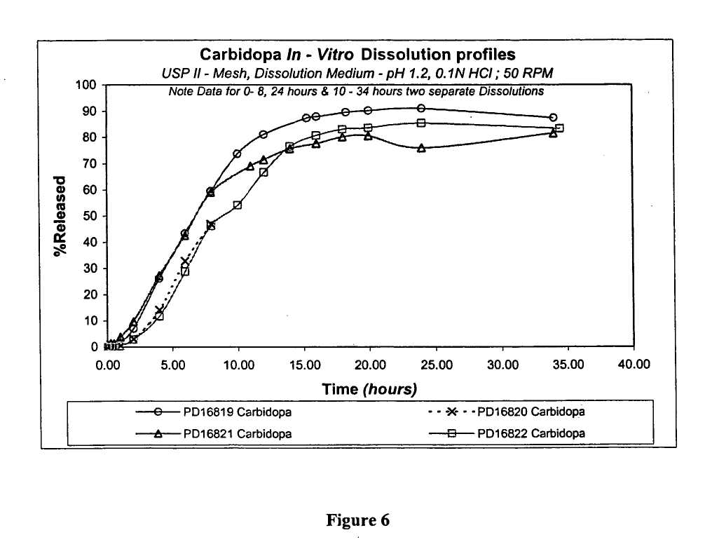

FIG. 2 shows the dissolution profile for the PEG 3350 100:25 mg levodopa:carbidopa (Granule Formulation 9/Tablet Formulation 19) dosage forms. FIG. 3 shows the dissolution profile for the Lutrol F68 100:25 mg levodopa:carbidopa (Granule Formulation 10/Tablet Formulation 20) dosage forms. FIG. 4 shows the dissolution profile for the Lutrol F68 200:50 mg levodopa:carbidopa (Granule Formulation 11/Tablet Formulation 21) dosage forms. FIG. 5 shows the dissolution profiles for levodopa release from 4 different dosage forms. FIG. 6 shows the dissolution profiles for carbidopa release from 4 different dosage forms.

Example 7-Inflation Time Course for Pouches

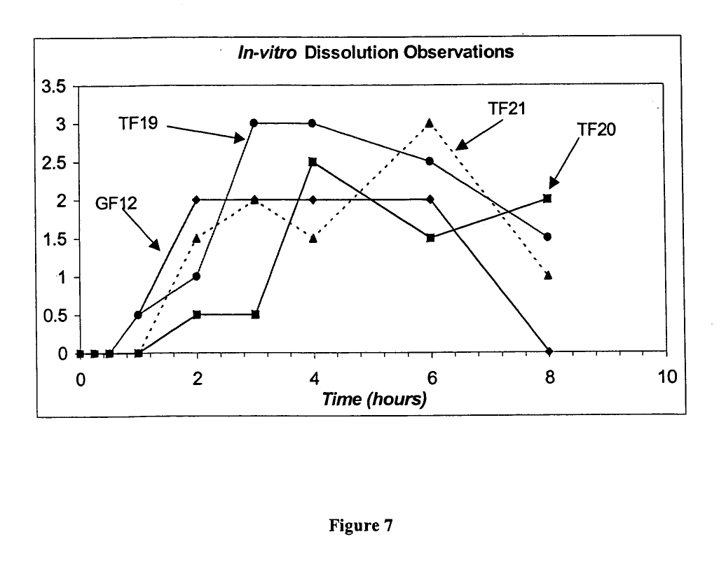

The inflation of the various dosage forms in the simulated gastric fluid described in Examples 3-5 were observed and rated on a semi-quantitative scale of 0 to 3, with 0 being not inflated, 1 being beginning to inflate, 2 being almost inflated and 3 being fully inflated. The results over an 8 hour time course are shown in FIG. 7.

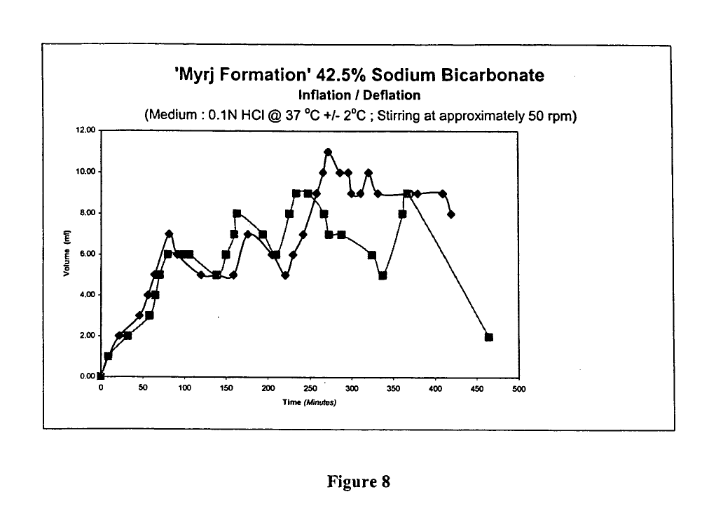

FIG. 8 shows an additional time courses of inflation-deflation of the dosage forms in simulated gastric fluid measuring the volume of gas retained in the pouches. The volume of gas generated was measured by placing the pouch in a customized sealable 1 liter Duran bottle with a graduated pipette protruding from the cap to allow measurement of change in height of the meniscus due to pouch expansion. Duplicate samples of each dosage form were measured every 1-5 minutes for 4 hours.

Example 8- Additional Formulations

Granules and tablets were prepared as described in Example 3-4 having the formulations shown in Tables 7-22.

TABLE 7
166:42 mg/g Levodopa:Carbidopa
granules without samarium oxide

| | | 300 g | |
|---|---|---|---|
| Raw material | Composition % | batch size g | 4 kg batch size g |
| Sodium bicarbonate | 71.08 | 213.24 | 2843.20 |
| Wetting agent* | 8.12 | 24.36 | 324.80 |
| Levodopa | 16.64 | 49.92 | 665.60 |
| Carbidopa | 4.16 | 12.48 | 166.40 |

*Wetting agent is Lutrol F68, Myrj 52P or PEG 3350

TABLE 8
166:42 mg/g Levodopa:Carbidopa
granules without samarium oxide

| | | 300 g | |
|---|---|---|---|
| Raw material | Composition % | batch size g | 4 kg batch size g |
| Sodium bicarbonate | 64.70 | 194.10 | 2588.00 |
| Wetting agent* | 14.50 | 43.50 | 580.00 |
| Levodopa | 16.64 | 49.92 | 665.60 |
| Carbidopa | 4.16 | 12.48 | 166.40 |

*Wetting agent is Lutrol F68, Myrj 52P or PEG 3350

TABLE 9
166:42 mg/g Levodopa:Carbidopa granules with samarium oxide

| | | 300 g | |
|---|---|---|---|
| Raw material | Composition % | batch size g | 4 kg batch size g |
| Sodium bicarbonate | 62.76 | 188.28 | 2510.40 |
| Wetting agent* | 8.12 | 24.36 | 324.80 |
| Samarium oxide | 8.32 | 24.96 | 332.80 |
| Levodopa | 16.64 | 49.92 | 665.60 |
| Carbidopa | 4.16 | 12.48 | 166.40 |

*Wetting agent is Lutrol F68, Myrj 52P or PEG 3350

TABLE 10
166:42 mg/g Levodopa:Carbidopa granules with samarium oxide

| | | 300 g | |
|---|---|---|---|
| Raw material | Composition % | batch size g | 4 kg batch size g |
| Sodium bicarbonate | 56.38 | 169.14 | 2255.20 |
| Wetting agent* | 14.50 | 43.50 | 580.00 |
| Samarium oxide | 8.32 | 24.96 | 332.80 |
| Levodopa | 16.64 | 49.92 | 665.60 |
| Carbidopa | 4.16 | 12.48 | 166.40 |

*Wetting agent is Lutrol F68, Myrj 52P or PEG 3350

TABLE 11
333:83 mg/g Levodopa:Carbidopa
granules without samarium oxide

| | | 300 g | |
|---|---|---|---|
| Raw material | Composition % | batch size g | 4 kg batch size g |
| Sodium bicarbonate | 50.27 | 150.81 | 2010.80 |
| Wetting agent* | 8.12 | 24.36 | 324.80 |
| Levodopa | 33.29 | 99.87 | 1331.60 |
| Carbidopa | 8.32 | 24.96 | 332.80 |

*Wetting agent is Lutrol F68, Myrj 52P or PEG 3350

TABLE 12
333:83 mg/g Levodopa:Carbidopa
granules without samarium oxide

| | | 300 g | |
|---|---|---|---|
| Raw material | Composition % | batch size g | 4 kg batch size g |
| Sodium bicarbonate | 43.89 | 131.67 | 1755.60 |
| Wetting agent* | 14.50 | 43.50 | 580.00 |
| Levodopa | 33.29 | 99.87 | 1331.60 |
| Carbidopa | 8.32 | 24.96 | 332.80 |

*Wetting agent is Lutrol F68, Myrj 52P or PEG 3350

TABLE 13
333:83 mg/g Levodopa:Carbidopa granules with samarium oxide

| | | 300 g | |
|---|---|---|---|
| Raw material | Composition % | batch size g | 4 kg batch size g |
| Sodium bicarbonate | 41.95 | 125.85 | 1678.00 |
| Wetting agent* | 8.12 | 24.36 | 324.80 |
| Samarium oxide | 8.32 | 24.96 | 332.80 |
| Levodopa | 33.29 | 99.87 | 1331.60 |
| Carbidopa | 8.32 | 24.96 | 332.80 |

*Wetting agent is Lutrol F68, Myrj 52P or PEG 3350

TABLE 14
333:83 mg/g Levodopa:Carbidopa granules with samarium oxide

| | | 300 g | |
|---|---|---|---|
| Raw material | Composition % | batch size g | 4 kg batch size g |
| Sodium bicarbonate | 35.57 | 106.71 | 1422.80 |
| Wetting agent* | 14.50 | 43.50 | 580.00 |
| Samarium oxide | 8.32 | 24.96 | 332.80 |
| Levodopa | 33.29 | 99.87 | 1331.60 |
| Carbidopa | 8.32 | 24.96 | 332.80 |

*Wetting agent is Lutrol F68, Myrj 52P or PEG 3350

TABLE 15
100:25 mg Levodopa:Carbidopa tablets without samarium oxide

| | | Quantity in tablet |
|---|---|---|
| Component | Composition % | mg |
| Wetting agent* | 8.00 | 52.00 |
| Sodium bicarbonate | 71.27 | 463.25 |
| Levodopa | 15.38 | 100.00 |
| Carbidopa | 3.85 | 25.00 |
| Aerosil 200 | 0.50 | 3.25 |
| Magnesium stearate | 1.00 | 6.50 |

*Wetting agent is Lutrol F68, Myrj 52P or PEG 3350

TABLE 16
100:25 mg Levodopa:Carbidopa tablets without samarium oxide

| | | Quantity in tablet |
|---|---|---|
| Component | Composition % | mg |
| Wetting agent* | 14.50 | 94.25 |
| Sodium bicarbonate | 64.77 | 421.00 |
| Levodopa | 15.38 | 100.00 |
| Carbidopa | 3.85 | 25.00 |
| Aerosil 200 | 0.50 | 3.25 |
| Magnesium stearate | 1.00 | 6.50 |

*Wetting agent is Lutrol F68, Myrj 52P or PEG 3350

TABLE 17
100:25 mg Levodopa:Carbidopa tablets with samarium oxide

| | | Quantity in tablet |
|---|---|---|
| Component | Composition % | mg |
| Wetting agent* | 8.00 | 52.00 |
| Sodium bicarbonate | 65.38 | 413.25 |
| Samarium oxide | 7.69 | 50.00 |
| Levodopa | 15.38 | 100.00 |
| Carbidopa | 3.85 | 25.00 |
| Aerosil 200 | 0.50 | 3.25 |
| Magnesium stearate | 1.50 | 6.50 |

*Wetting agent is Lutrol F68, Myrj 52P or PEG 3350

TABLE 18
100:25 mg Levodopa:Carbidopa tablets with samarium oxide

| | | Quantity in tablet |
|---|---|---|
| Component | Composition % | mg |
| Wetting agent* | 14.50 | 94.25 |
| Sodium bicarbonate | 57.08 | 371.00 |
| Samarium oxide | 7.69 | 50.00 |
| Levodopa | 15.38 | 100.00 |
| Carbidopa | 3.85 | 25.00 |
| Aerosil 200 | 0.50 | 3.25 |
| Magnesium stearate | 1.50 | 6.50 |

*Wetting agent is Lutrol F68, Myrj 52P or PEG 3350

TABLE 19
200:50 mg Levodopa:Carbidopa tablets without samarium oxide

| | | Quantity in tablet |
|---|---|---|
| Component | Composition % | mg |
| Wetting agent* | 8.00 | 52.00 |
| Sodium bicarbonate | 52.04 | 338.25 |
| Levodopa | 30.77 | 200.00 |
| Carbidopa | 7.69 | 50.00 |
| Aerosil 200 | 0.50 | 3.25 |
| Magnesium stearate | 1.00 | 6.50 |

*Wetting agent is Lutrol F68, Myrj 52P or PEG 3350

TABLE 20
200:50 mg Levodopa:Carbidopa tablets without samarium oxide

| | | Quantity in tablet |
|---|---|---|
| Component | Composition % | mg |
| Wetting agent* | 14.50 | 94.25 |
| Sodium bicarbonate | 45.54 | 296.00 |
| Levodopa | 30.77 | 200.00 |
| Carbidopa | 7.69 | 50.00 |
| Aerosil 200 | 0.50 | 3.25 |
| Magnesium stearate | 1.00 | 6.50 |

*Wetting agent is Lutrol F68, Myrj 52P or PEG 3350

TABLE 21
200:50 mg Levodopa:Carbidopa tablets with Samarium oxide

| | | Quantity in tablet |
|---|---|---|
| Component | Composition % | mg |
| Wetting agent* | 8.00 | 52.00 |
| Sodium bicarbonate | 44.35 | 288.25 |
| Samarium oxide | 7.69 | 50.00 |
| Levodopa | 30.77 | 200.00 |
| Carbidopa | 7.69 | 50.00 |
| Aerosil 200 | 0.50 | 3.25 |
| Magnesium stearate | 1.50 | 6.50 |

*Wetting agent is Lutrol F68, Myrj 52P or PEG 3350

TABLE 22
200:50 mg Levodopa:Carbidopa tablets with Samarium oxide

| | | Quantity in tablet |
|---|---|---|
| Component | Composition % | mg |
| Wetting agent* | 14.50 | 94.25 |
| Sodium bicarbonate | 37.85 | 246.00 |
| Samarium oxide | 7.69 | 50.00 |
| Levodopa | 30.77 | 200.00 |
| Carbidopa | 7.69 | 50.00 |
| Aerosil 200 | 0.50 | 3.25 |
| Magnesium stearate | 1.50 | 6.50 |

*Wetting agent is Lutrol F68, Myrj 52P or PEG 3350

The subject matter claimed is:

1. A gastro-retentive dosage form of levodopa for oral administration to a patient in need thereof, said dosage form comprising
(a) a tablet comprising a therapeutically effective amount of levodopa, a binder, and a pharmaceutically-acceptable gas-generating agent capable of releasing carbon dioxide upon contact with gastric juice, and
(b) an expandable, hydrophilic, water-permeable and substantially gas-impermeable, membrane surrounding the tablet, wherein the membrane expands as a result of the release of carbon dioxide from the gas-generating agent upon contact with the gastric juice, whereby the dosage form becomes too large to pass into the patient's pyloric sphincter.

2. The dosage form of claim 1, further comprising a covering for containing the dosage form, wherein the covering disintegrates upon contact with gastric fluid.

3. The dosage form of claim 2, wherein the covering is a dry-fill capsule.

4. The dosage form of claim 1, wherein the levodopa is present in an amount of about 10% to about 50% of the total tablet weight.

5. The dosage form of claim 1, wherein the tablet further comprises carbidopa.

6. The dosage form of claim 1, wherein the membrane comprises polyvinyl alcohol.

7. The dosage form of claim 6, wherein the polyvinyl alcohol is present in the membrane at between 40% and 85%.

8. The dosage form of claim 1, wherein the tablet comprises levodopa and carbidopa in a weight ratio of between about 4-to-1 and about 10-to-1 levodopa to carbidopa.

9. The dosage form of claim 1, wherein the gas-generating agent is selected from the group consisting of sodium bicarbonate, sodium carbonate, sodium glycine carbonate, potassium carbonate, calcium carbonate, magnesium carbonate and mixtures thereof.

10. The dosage form of claim 9, wherein the gas-generating agent is sodium bicarbonate.

11. The dosage form of claim 1, wherein the binder is selected from the group consisting of a polyoxyethylene stearate, a poloxamer, a polyethylene glycol, a glycerol palmitostearate, a glyceryl monostearate, a methylcellulose and a polyvinyl pyrrolidone.

12. The dosage form of claim 11, wherein the binder is selected from the group consisting of Myrj 52, Lutrol F68, PEG 3350, a methylcellulose and a polyvinyl pyrrolidone.

13. A method of making a gastro-retentive dosage form of levodopa, which method comprises
(a) forming a tablet comprising levodopa, a binder and a pharmaceutically-acceptable gas-generating agent,
(b) surrounding the tablet with an expandable, hydrophilic, water-permeable and substantially gas-impermeable membrane, and
(c) sealing the membrane to retard the escape of gas from within the sealed membrane.

14. The method of claim 13, further comprising the step of encapsulating the sealed membrane within a covering that disintegrates without delay upon contact with gastric fluid.

15. The method of claim 14, wherein said covering is a dry-fill capsule.

16. The method of claim 13, wherein the tablet formed in (a) also comprises carbidopa.

17. The method of claim 13, wherein the levodopa is present in an amount of about 10% to about 50% of the total weight of the tablet formed in (a).

18. The method of claim 13, wherein the membrane comprises polyvinyl alcohol.

19. The method of claim 18, wherein polyvinyl alcohol is present in the membrane at between 40% and 85%.

20. The method of claim 13, wherein the tablet formed in (a) comprises levodopa and carbidopa in a weight ratio of between about 4-to-1 and about 10-to-1 levodopa to carbidopa.

21. The method of claim 13, wherein the gas-generating agent is selected from the group consisting of sodium bicarbonate, sodium carbonate, sodium glycine carbonate, potassium carbonate, calcium carbonate, magnesium carbonate, and mixtures thereof.

22. The method of claim 21, wherein the gas-generating agent is sodium bicarbonate.

23. The method of claim 13, wherein the binder is selected from the group consisting of a polyoxyethylene stearate, a poloxamer, a polyethylene glycol, a glycerol palmitostearate, a glyceryl mono stearate, a methylcellulose, and a polyvinyl pyrrolidone.

24. The method of claim 23, wherein the binder is selected from the group consisting of Myrj52, Lutrol F68, PEG 3350, Precirol ATO5, a methylcellulose, and a polyvinyl pyrrolidone.

25. The method of claim 24, wherein the binder is selected from the group consisting of Myrj52, Lutrol F68 and PEG 3350.

26. The method of claim 13, wherein the forming step comprises fluid bed granulation or melt granulation.

27. A method of treating a patient suffering from Parkinson's disease comprising orally administering to said patient the gastro-retentive dosage form according to claim 1.

28. An article of manufacture comprising the dosage form according to claim 1, packaging material containing the dosage form and a label or insert indicating instructions for use of the dosage form for treatment of Parkinson's disease.
